(19) United States
(12) Patent Application Publication
(10) Pub. No.: US 2007/0015805 A1
(43) Pub. Date: Jan. 18, 2007

(54) Process for preparing 3-arylmethylthio- and 3-heteroarylmethylthio-4,5-dihydroisoxazoline derivatives

(75) Inventors: Wolfgang Schaper, Diedorf (DE); Hendrik Helmke, Liederbach (DE)

Correspondence Address: STERNE, KESSLER, GOLDSTEIN & FOX PLLC, 1100 NEW YORK AVENUE, N.W., WASHINGTON, DC, 20005, US

(73) Assignee: Bayer CropScience GmbH, Frankfurt (DE)

(21) Appl. No.: 11/476,693

(22) Filed: Jun. 29, 2006

(30) Foreign Application Priority Data
Jul. 6, 2005 (DE) 102005031583.6

(51) Int. Cl. A61K 31/42; C07D 261/10

(52) U.S. Cl. 514/378; 548/243

(57) ABSTRACT

The invention relates to a process for preparing 3-arylmethylthio- and 3-heteroarylmethylthio-4,5-dihydroisoxazoline derivatives of the formula (I) 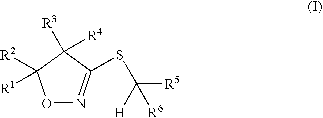 by a one-pot process, by reacting corresponding arylmethyl- and heteroarylmethylisothiuronium salts, in the presence of an aqueous alkali metal or alkaline earth metal base, with a isoxazoline derivative to give the corresponding 3-arylmethylthio- and 3-heteroarylmethylthio-4,5-dihydroisoxazoline derivatives.

The invention relates to a process for preparing 3-arylmethylthio- and 3-heteroarylmethylthio-4,5-dihydroisoxazoline derivatives by a one-pot process, by reacting corresponding arylmethyl- and heteroarylmethylisothiuronium salts, in the presence of an aqueous alkali metal or alkaline earth metal base, with a isoxazoline derivative to give the corresponding 3-arylmethylthio- and 3-heteroarylmethylthio-4,5-dihydroisoxazoline derivatives.

The literature discloses that isoxazoline derivatives of the formula (1) in which R^{a} and R^{b} are preferably optionally substituted alkyl radicals, R^{c}, R^{d} and R^{e} are preferably each hydrogen, R^{f} is an optionally substituted aryl or heteroaryl radical and X is hydrogen, and also their halogenmethylsulfinyl and halogenmethylsulfonyl analogs for which X is halogen, have interesting herbicidal action (cf., for example, WO 2001 012613, WO 2002 062770, WO 2003 000686 and WO 2003 010165).

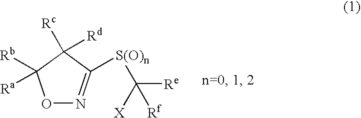

To prepare the abovementioned compounds, according to the processes known to date, the corresponding thioether of the formula (1) where n=0 is first prepared in each case, and is then converted further to the oxidized and halogenated derivatives.

The processes corresponding to the prior art for preparing the thioethers of the formula (1) (n=0) (for example WO 2001 012613, WO 2002 062770, WO 2003 000686 and WO 2003 010165) utilize:
(a) the hydrolysis of an isothiuronium salt of the formula (2) in which R^{e} and R^{f} are each as defined above for formula (1) and Lg is a leaving group to a mercaptan of the formula (3) in which R^{e} and R^{f} are each as defined above for formula (1) and its subsequent reaction with an isoxazoline of the formula (4) in which R^{a}, R^{b}, R^{c} and R^{d} are each as defined above for formula (1) and Lg′ is a leaving group; 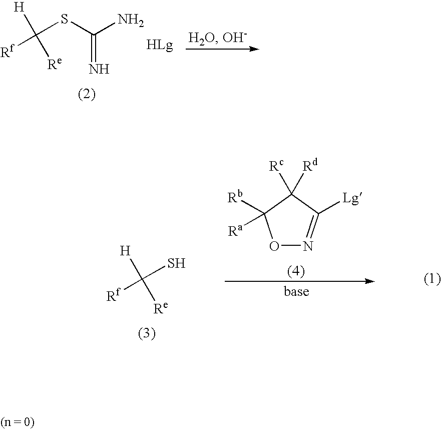
   or
(b) the conversion of an isoxazoline of the formula (4) via three intermediate steps to a 3-mercaptoisoxazoline of the formula (5) in which R^{a}, R^{b}, R^{c} and R^{d} are each as defined above for formula (1) and its subsequent alkylation with an aryl- or heteroarylmethyl derivative of the formula (6) in which R^{e} and R^{f} are each as defined above for formula (1) and Lg is a leaving group.

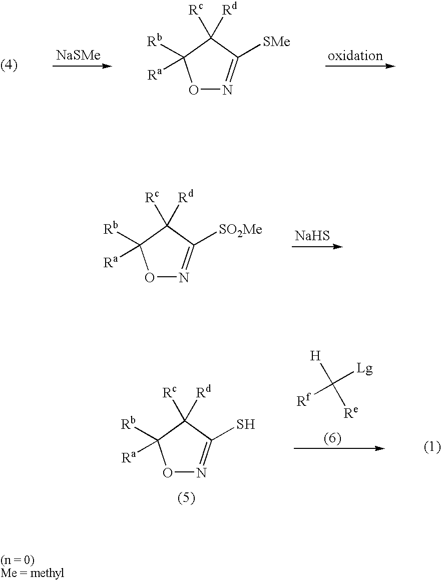

The disadvantage of synthesis variant (a) is the handling of the mercaptans (3) which are for the most part toxic, malodorous and oxidation-sensitive, and the disadvantage of variant (b) is the high number of stages and the associated unsatisfactory overall yield.

It is thus an object of the invention to provide a synthesis process for the abovementioned thioethers of the formula (1) and further analogs, which avoids the abovementioned disadvantages of the processes according to (a) or (b).

The present invention thus relates to a process for preparing 3-arylmethylthio- and 3-heteroarylmethylthio-4,5-dihydroisoxazoline derivatives of the formula (I) 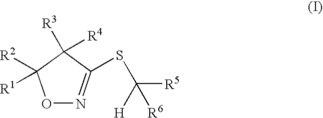
where
R¹, R² are each independently hydrogen, (C₁-C₆)-alkyl, (C₂-C₆)-alkenyl, (C₂-C₆)-alkynyl or (C₃-C₈)-cycloalkyl, where each of the (C₁-C₆)-alkyl, (C₂-C₆)-alkenyl, (C₂-C₆)-alkynyl or (C₃-C₈)-cycloalkyl radicals is unsubstituted or substituted by one or more identical or different radicals from the group of halogen, cyano, (C₃-C₈)-cycloalkyl or else by —OR⁷ or —S(O)ₘR⁷ where m=0, 1 or 2, and R⁷ corresponds to a (C₁-C₆)-alkyl, (C₃-C₆)-alkenyl, (C₃-C₆)-alkynyl, (C₃-C₈)-cycloalkyl, each of which is unsubstituted or substituted by one or more identical or different radicals from the group of halogen and cyano,
   or else R¹ and R² together form a spiro linkage composed of from 3 to 8 carbon atoms together with the carbon atom to which they are both bonded,
R³, R⁴ are each hydrogen, (C₁-C₆)-alkyl, (C₂-C₆)-alkenyl, (C₂-C₆)-alkynyl or (C₃-C₈)-cycloalkyl, where the aforementioned alkyls, cycloalkyls, alkenyls or alkynyls are optionally substituted by one or more identical or different radicals from the group of halogen, cyano, (C₃-C₈)-cycloalkyl, (C₁-C₆)-alkoxy, (C₁-C₆)-haloalkoxy or (C₁-C₆)-alkylthio,
   or else R³ and R⁴ together form a spiro linkage composed of from 3 to 8 carbon atoms together with the carbon atom to which they are both bonded,
   or R¹ and R³ together with the carbon atoms to which they are bonded form a ring structure consisting of 5-8 carbon atoms,
R⁵ is unsubstituted or substituted aryl, preferably having from 6 to 14 carbon atoms, or unsubstituted or substituted heteroaryl having preferably from 1 to 9 carbon atoms and one or more heteroatoms, preferably having from 1 to 4 heteroatoms, in particular having from 1 to 3 heteroatoms from the group of N, O and S, where each of the carbocyclic or heterocyclic radicals above is optionally substituted by OH, halogen, cyano, (C₁-C₆)-alkyl, (C₁-C₆)-haloalkyl, (C₂-C₆)-alkenyl, (C₂-C₆)-alkynyl, (C₃-C₈)-cycloalkyl, (C₃-C₆)-cycloalkenyl, mono-(C₁-C₆)-alkylamino, di-((C₁-C₆)-alkyl)amino, N-(C₁-C₆)-alkanoyl)amino, (C₁-C₆)-alkoxy, (C₁-C₆)-haloalkoxy, (C₃-C₆)-alkenyloxy, (C₃-C₆)-alkynyloxy, (C₃-C₆)-cycloalkoxy, (C₄-C₆)-cycloalkenyloxy, (C₁-C₆)-alkylthio, (C₁-C₆)-haloalkylthio, (C₃-C₆)-cycloalkylthio, (C₃-C₆)-alkenylthio, (C₄-C₆)-cycloalkenylthio, (C₃-C₆)-alkynylthio, (C₁-C₆)-alkanoyl, (C₂-C₆)-alkenylcarbonyl, (C₂-C₆)-alkynylcarbonyl, arylcarbonyl, (C₁-C₆)-alkylsulfinyl, (C₁-C₆)-alkylsulfonyl, (C₁-C₆)-haloalkylsulfinyl or (C₁-C₆)-haloalkylsulfonyl,
   and
R⁶ is hydrogen or (C₁-C₆)-alkyl
by starting from arylmethyl- and heteroarylmethylisothiuronium salts of the formula (II) 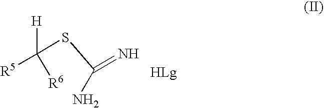
in which R⁵ and R⁶ are each as defined above for the formula (I) and Lg is a leaving group,
and reacting it in a one-pot process in the presence of an aqueous alkali metal or alkaline earth metal base with an isoxazoline derivative of the formula (IV) 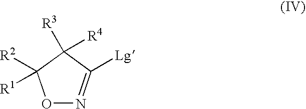
in which R¹, R², R³ and R⁴ are each as defined above for the formula (I) and Lg′ is a leaving group
to give the target compounds, i.e. the corresponding 3-arylmethylthio- and 3-heteroarylmethylthio-4,5-dihydroisoxazolines of the formula (I).

Compounds of the formula (II) can be obtained by reacting an alkylating agent of the formula R⁵R⁶CHLg where R⁵ and R⁶ are each as defined above for the formula (I) and Lg is a leaving group with thiourea.

The use of isothiuronium salts in a one-pot reaction for hydrolyzing the isothiuronium salt and converting the mercaptan formed as an intermediate in an exchange reaction is described in DE 3942946 for another reaction scheme.

In general, the reaction is illustrated by the formula scheme which follows:

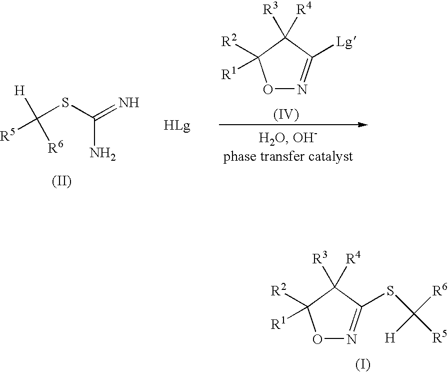

The mercaptan of the formula (III) which is formed as an intermediate under the reaction conditions in which R⁵ and R⁶ are each as defined above for the formula (I) 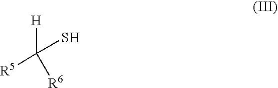
is scavenged in situ immediately by the isoxazoline of the formula (IV). The handling of the mercaptan with the abovementioned unpleasant properties is avoided in the process according to the invention. In addition, the preparation is shortened by one stage compared to variant (a) of the prior art.

Compared to variant (b) of the prior art, the process according to the invention has a number of stages reduced by 2.

Preferred leaving groups Lg are chlorine, bromine, iodine or sulfonate groups, such as methane-, trifluoromethane-, ethane-, benzene- or toluenesulfonate.

Preferred leaving groups Lg′ are chlorine, bromine or sulfonate groups such as methane-, trifluoromethane-, ethane-, benzene- or toluenesulfonate, or methylsulfonyl, but particularly chlorine.

When the process according to the invention is employed, preference is given to compounds of the formula (I) in which R¹ and R² are each independently (C₁-C₄)-alkyl, (C₂-C₃)-alkenyl, (C₂-C₃)-alkynyl, (C₃-C₆)-cycloalkyl, where each of the (C₁-C₄)-alkyl, (C₂-C₃)-alkenyl, (C₂-C₃)-alkynyl, (C₃-C₆)-cycloalkyl radicals are optionally substituted by one or more radicals from the group of halogen, cyano or (C₃-C₆)-cycloalkyl.

When the process according to the invention is employed, particular preference is given to compounds of the formula (I) in which R¹ and R² are each independently (C₁-C₄)-alkyl or (C₁-C₄)-haloalkyl.

When the process according to the invention is employed, preference is further given to compounds of the formula (I) in which R¹ and R² are each independently methyl or ethyl which have in turn optionally each independently been mono- or polyhalogenated, preferably -chlorinated or -fluorinated.

Among the halogenated radicals, preference is given to chloromethyl and fluoromethyl, very particular preference to chloromethyl.

When the process according to the invention is employed, preference is further given to compounds of the formula (I) in which R³ and R⁴ are each independently hydrogen or (C₁-C₄)-alkyl.

When the process according to the invention is employed, particular preference is given to compounds of the formula (I) in which R³ and R⁴ each correspond to hydrogen.

When the process according to the invention is employed, preference is further given to compounds of the formula (I) in which R⁵ is an unsubstituted or substituted aryl preferably having from 6 to 10 carbon atoms or unsubstituted or substituted heteroaryl having preferably from 1 to 9 carbon atoms, preferably from 3 to 5 carbon atoms, with from 1 to 3 heteroatoms, preferably with one or two identical or different heteroatoms, from the group of N, O and S, where each of the above carbocyclic or heterocyclic radicals is optionally substituted by halogen, cyano, (C₁-C₃)-alkyl, (C₁-C₃)-haloalkyl, (C₂-C₄)-alkenyl, (C₂-C₄)-alkynyl, (C₃-C₆)-cycloalkyl, (C₃-C₆)-cycloalkenyl, (C₁-C₄)-alkoxy, (C₁-C₄)-haloalkoxy, (C₃-C₄)-alkenyloxy, (C₃-C₄)-alkynyloxy, (C₃-C₆)-cycloalkoxy, (C₄-C₆)-cycloalkenyloxy, (C₁-C₄)-alkylthio, (C₁-C₄)-haloalkylthio, (C₃-C₆)-cycloalkylthio, (C₃-C₄)-alkenylthio, (C₄-C₆)-cycloalkenylthio, (C₃-C₄)-alkynylthio, (C₁-C₄)-alkanoyl, (C₂-C₆)-alkenylcarbonyl, (C₂-C₆)-alkynylcarbonyl, arylcarbonyl, (C₁-C₄)-alkylsulfinyl, (C₁-C₄)-alkylsulfonyl, (C₁-C₄)-haloalkylsulfinyl or (C₁-C₄)-haloalkylsulfonyl.

When the process according to the invention is employed, preference is equally given to compounds of the formula (I) in which R⁵ is an unsubstituted or substituted aryl preferably having from 6 to 10 carbon atoms or unsubstituted or substituted heteroaryl having preferably from 3 to 5 carbon atoms with from 1 to 3 heteroatoms, preferably with one or two identical or different heteroatoms, from the group of N, O and S, where each of the above carbocyclic or heterocyclic radicals is optionally substituted by one or more identical or different radicals from the group of halogen, cyano, ethyl, methyl, haloethyl, halomethyl, halomethoxy or haloethoxy.

When the process according to the invention is employed, particular preference is given to compounds of the formula (I) in which R⁵ corresponds to a substituted or unsubstituted phenyl, naphthyl, thienyl, furyl, pyrazolyl, pyridinyl, pyrimidinyl, pyrazinyl, pyridazinyl, imidazolyl, isothiazolyl, thiazolyl or oxazolyl, most preferably to a phenyl or pyrazolyl, each of which, in the case of substitution, is preferably substituted by one or more identical or different radicals from the group of halogen, cyano, ethyl, methyl, halomethoxy or halomethyl, where preference is given among the halogens to chlorine and fluorine, and in the case of halomethoxy and halomethyl very particularly to fluorine.

The isoxazolines (IV) in which Lg′ is defined as a leaving group, for example halogen, SO₂Me, SOMe or similar, used as starting materials in the process according to the invention, are familiar to those skilled in the art and are described, inter alia, in:
Rohloff, J. C.; Robinson, J. I.; Gardner J. O.; Tetrahedron Lett. (1992) 33 3113, WO 2001012613 and WO 2002 062770.

The preparation of isothiuronium salts from the corresponding alkylating agents and thiourea is effected by literature processes, advantageously by reacting a corresponding alkylating agent of the formula R⁵R⁶CHLg where R⁵,R⁶ and Lg are each as specified above with a equimolar amount of thiourea in an inert solvent such as lower alcohols, for example methanol, ethanol or isopropanol; hydrocarbons, for example benzene or toluene; halogenated hydrocarbons, for example dichloromethane or chloroform; or ether derivatives, for example methyl tert-butyl ether, tetrahydrofuran or dioxane, at temperatures between 0° and 150° C., preferably from 20° to 100° C.

The compounds of isothiuronium salts of the formula (II) obtained in many cases by crystallization, generally without further purification steps, are reacted in the process according to the invention with equimolar amounts of the compounds of the formula (IV) under phase transfer conditions-with vigorous stirring.

Preference is given here to working in a biphasic system, in which case, as well as an aqueous, strongly basic alkali metal or alkaline earth metal hydroxide solution, preferably sodium or potassium hydroxide, with at least two equivalents of the base, the organic phase is an inert solvent such as tetrahydrofuran, diethyl ether, acetonitrile, pentane, hexane, benzene, toluene, xylene, chlorobenzene, dichloromethane, chloroform, carbon tetrachloride, nitrobenzene or mixtures of these solvents.

It is also possible to use the more valuable reactant of the formula (II) or of the formula (IV) in each case in a slight deficiency.

Suitable phase transfer catalysts are quaternary ammonium or phosphonium salts, and also crown ethers, cryptands or polyethylene glycols. Examples of such catalysts can be found, for example, in W. P. Weber, G. W. Gokel; Phase Transfer Catalysis in Organic Synthesis, Springer-Verlag, Berlin 1977 or E. V. Dehmlow, S. S. Dehmlow, Phase Transfer Catalysis, Second Ed. Verlag Chemie, Weinheim 1983.

The reaction of the isothiuronium salts (II) with the isoxazolines (IV) is effected within a temperature range of from −10° to 150° C. under the conditions of a phase transfer-catalyzed reaction.

The reactants and the catalyst are preferably stirred vigorously under protective gas atmosphere at temperatures of from 20° to 100° C.

The compounds obtained can, if required, be oxidized and/or halogenated by reactions known to those skilled in the art.

The synthesis examples which follow illustrate the process according to the invention. Percentages are based on the weight.

### SYNTHESIS EXAMPLE A

### Preparation of 3-(2,6-difluorobenzylthio)-5-ethyl-5-methyl-4,5-dihydroisoxazole

2.0 g (14 mmol) of 3-chloro-5-ethyl-5-methyl-4,5-dihydroisoxazole (preparation according to WO 2001 012613) and 3.84 g (14 mmol) of 2,6-difluorobenzylisothiuronium bromide (prepared by reacting equimolar amounts of 2,6-difluorobenzyl bromide and thiourea in ethanol) were stirred vigorously under argon at room temperature with 1.22 g (4 mmol) of tetra-n-butylammonium bromide in a mixture of 100 ml of toluene and 28 g of 50% sodium hydroxide solution for 6 hours. After dilution with water, the organic phase was dried and concentrated. For purification, the residue was chromatographed on silica gel (4:1 ethyl acetate/heptane).

1.98 g (51.2% of theory) of product were obtained as a colorless oil. ¹H NMR (300 MHz, CCCl₃): (CDCl₃): 2.80 (AB, 2H, isoxazoline CH₂); 4.36 (s, 2H, CH₂S)

### SYNTHESIS EXAMPLE B

### Preparation of 5,5-dimethyl-3-(2-trifluoromethoxybenzylthio)-4,5-dihydroisoxazole

0.81 g (3 mmol) of tetra-n-butylammonium bromide, 1.20 g (9 mmol) of 3-chloro-5,5-dimethyl-4,5-dihydroisoxazole (preparation according to WO 2001 012613) and 2.98 g (9 mmol) of 2-trifluoromethoxybenzylisothiuronium bromide (prepared by heating equimolar amounts of thiourea and 2-trifluoromethoxybenzyl bromide in ethanol) were added under argon to a mixture of 50 ml of toluene and 21 g of 50% aqueous sodium hydroxide solution. The mixture was stirred vigorously at room temperature for 6 hours. After dilution with water, the toluene phase was removed, the water phase was once again extracted by stirring with toluene and the combined organic phases were dried and concentrated. For purification, the crude product was chromatographed on silica gel (4:1 heptane/ethyl acetate).

1.68 g of product (58% of theory) were obtained as a colorless oil.

NMR (300 MHz, CDCl₃): 1.21 (s, 6H, 2CH₃); 2.78 (s, 2H, isoxazoline CH₂); 4.24 (s, 2H, SCH₂); 7.20-7.35 (m, 3H, phenyl H); 7.56 (dd, 1H, phenyl H)

### SYNTHESIS EXAMPLE C

### Preparation of 3-({[5-(difluoromethoxy)-1-methyl-3-(trifluoromethyl)-1H-pyrazol-4-yl]methyl}thio)-5,5-dimethyl-4,5-dihydroisoxazole

4.05 g (13 mmol) of tetra-n-butylammonium bromide, 13.67 g (35 mmol) of [5-(difluoromethoxy)-1-methyl-3-(trifluoromethyl)-1H-pyrazol-4-yl]methyl imidothiocarbamate hydrobromide (WO 2004 013106) were added under an argon atmosphere to a mixture of 74.000 g of 50% aqueous sodium hydroxide solution and 100 ml of toluene. A solution of 6.000 g (45 mmol) of 3-chloro-5,5-dimethyl-4,5-dihydroisoxazole in a little toluene was added dropwise thereto at room temperature with vigorous stirring. The batch was stirred at room temperature for a further two hours and diluted with water. The mixture was extracted three times by stirring with ethyl acetate and the combined organic phases were dried and concentrated. For purification, the crude product was chromatographed on silica gel (3:3 heptane/ethyl acetate).

8.28 g (64.9% of theory) of product were obtained as a colorless oil. NMR (400 MHz, CDCl₃): 1.41 (s, 6H, 2CH₃); 2.78 (s, 2H, isoxazoline CH₂); 3.82 (s, 3H, NCH₃); 4.18 (s, 2H, SCH₂); 6.73 (tr, 1H, OCF₂H)

The compounds described in Table A below are obtained according to or analogously to the above-described Synthesis Examples A to C.

In the tables:

TABLE A

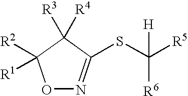

| Ex. | | | | | | |
|---|---|---|---|---|---|---|
| No. | R¹ | R² | R³ | R⁴ | R⁵ | R⁶ |
| 1 | Me | Me | H | H | Ph | H |
| 2 | Me | Et | H | H | Ph | H |
| 3 | Me | Me | H | H | 2,6-F₂-Ph | H |
| 4 | Me | Me | H | H | 2,5-F₂-Ph | H |
| 5 | Me | Et | H | H | 2,5-F₂-Ph | H |
| 6 | Me | Me | H | H | 2,5-Me₂-Ph | H |
| 7 | Me | Et | H | H | 2,5-Me₂-Ph | H |
| 8 | Me | Me | H | H | 2-F-Ph | H |
| 9 | Me | Et | H | H | 2-F-Ph | H |
| 10 | Me | Et | H | H | 2-F-4-CF₃-Ph | H |
| 11 | Me | Et | H | H | 2,4,5-F₃-Ph | H |
| 12 | Me | Et | H | H | 2,4,6-Me₃-Ph | H |
| 13 | Me | Me | H | H | 2,4-Cl₂-Ph | H |
| 14 | Me | Et | H | H | 2-Cl-Ph | H |
| 15 | Me | Et | H | H | 2,4-Cl₂-Ph | H |
| 16 | Me | Et | H | H | 2-Me-Ph | H |
| 17 | Me | Et | H | H | 2,5-Cl₂-Ph | H |
| 18 | Me | Me | H | H | 2,5-Cl₂-Ph | H |
| 19 | Me | Et | H | H | 2-F-5-Cl-Ph | H |
| 20 | Me | Me | H | H | 2-F-5-Cl-Ph | H |
| 21 | Me | Et | H | H | 3,5-Me₂-isoxazol-4-yl | H |
| 22 | Me | Me | H | H | 3,5-Me₂-isoxazol-4-yl | H |
| 23 | Me | Et | H | H | 2-Cl-5-F-Ph | H |
| 24 | Me | Me | H | H | 2-Cl-5-F-Ph | H |
| 25 | Me | Et | H | H | 3-F-Ph | H |
| 26 | Me | Et | H | H | 2-CF₃-Ph | H |
| 27 | Me | Et | H | H | 3-CF₃-Ph | H |
| 28 | Me | Et | H | H | 4-CF₃-Ph | H |
| 29 | Me | Et | H | H | 3,4-Cl₂-Ph | H |
| 30 | Me | Et | H | H | 1-naphthyl | H |
| 31 | Me | Me | H | H | 5-Cl-1,3-Me₂-pyrazol-4-yl | H |
| 32 | Me | Et | H | H | 5-Cl-1,3-Me₂-pyrazol-4-yl | H |
| 33 | Me | Et | H | H | 5-Cl-1-Me-3-CF₃-pyrazol-4-yl | H |
| 34 | Me | Me | H | H | 2-OCF₂H-Ph | H |
| 35 | Me | Et | H | H | 5-OCF₂H-1-Me-3-CF₃-pyrazol-4-yl | H |
| 36 | Me | Me | H | H | 2,4,6-F₃-Ph | H |
| 37 | Me | CH₂Cl | H | H | 2,6-F₂-Ph | H |

Me = methyl
Et = ethyl
Ph = phenyl

The above examples were all prepared according to the claimed process.

1. A process for preparing a 3-arylmethylthio- or a 3-heteroarylmethylthio-4,5-dihydroisoxazoline derivative of the formula (I) 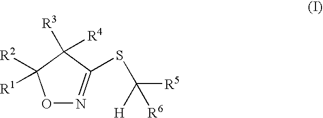
where
R¹, R² are each independently hydrogen, (C₁-C₆)-alkyl, (C₂-C₆)-alkenyl, (C₂-C₆)-alkynyl or (C₃-C₈)-cycloalkyl, where each of the (C₁-C₆)-alkyl, (C₂-C₆)-alkenyl, (C₂-C₆)-alkynyl or (C₃-C₈)-cycloalkyl radical is unsubstituted or substituted by one or more identical or different radicals selected from the group consisting of halogen, cyano, (C₃-C₈)-cycloalkyl, —OR⁷ and —S(O)ₘR⁷ where m=0, 1 or 2, and R⁷ is (C₁-C₆)-alkyl, (C₃-C₆)-alkenyl, (C₃-C₆)-alkynyl, or (C₃-C₈)-cycloalkyl, each of which is unsubstituted or substituted by one or more identical or different radicals selected from the group consisting of halogen and cyano,
or R¹ and R² together with the carbon atom to which they are both attached form a spiro linkage composed of from 3 to 8 carbon atoms,
R³, R⁴ are each independently hydrogen, (C₁-C₆)-alkyl, (C₂-C₆)-alkenyl, (C₂-C₆)-alkynyl or (C₃-C₈)-cycloalkyl, where the (C₁-C₆)-alkyl, (C₂-C₆)-alkenyl, (C₂-C₆)-alkynyl or (C₃-C₈)-cycloalkyl group is optionally substituted by one or more identical or different radicals selected from the group consisting of halogen, cyano, (C₃-C₈)-cycloalkyl, (C₁-C₆)-alkoxy, (C₁-C₆)-haloalkoxy and (C₁-C₆)-alkylthio,
or R³ and R⁴ together with the carbon atom to which they are attached form a spiro linkage composed of from 3 to 8 carbon atoms,
or R¹ and R³ together with the carbon atoms to which they are attached form a ring structure consisting of 5-8 carbon atoms,
R⁵ is unsubstituted or substituted aryl, or unsubstituted or substituted heteroaryl, where the aryl or heteroaryl group is optionally substituted by OH, halogen, cyano, (C₁-C₆)-alkyl, (C₁-C₆)-haloalkyl, (C₂-C₆)-alkenyl, (C₂-C₆)-alkynyl, (C₃-C₈)-cycloalkyl, (C₃-C₆)-cycloalkenyl, mono-(C₁-C₆)-alkylamino, di-((C₁-C₆)-alkyl)amino, N-((C₁-C₆)-alkanoyl)-amino, (C₁-C₆)-alkoxy, (C₁-C₆)-haloalkoxy, (C₃-C₆)-alkenyloxy, (C₃-C₆)-alkynyloxy, (C₃-C₆)-cycloalkoxy, (C₄-C₆)-cycloalkenyloxy, (C₁-C₆)-alkylthio, (C₁-C₆)-haloalkylthio, (C₃-C₆)-cycloalkylthio, (C₃-C₆)-alkenylthio, (C₄-C₆)-cycloalkenylthio, (C₃-C₆)-alkynylthio, (C₁-C₆)-alkanoyl, (C₂-C₆)-alkenylcarbonyl, (C₂-C₆)-alkynylcarbonyl, arylcarbonyl, (C₁-C₆)-alkylsulfinyl, (C₁-C₆)-alkylsulfonyl, (C₁-C₆)-haloalkylsulfinyl or (C₁-C₆)-haloalkylsulfonyl, and
R⁶ is hydrogen or (C₁-C₆)-alkyl, comprising
allowing an arylmethyl- or a heteroarylmethylisothiuronium salt of the formula (II) 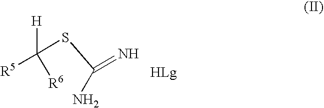
in which R⁵ and R⁶ are each as defined above for the formula (I) and Lg is a leaving group,
to react in a one-pot process in the presence of an aqueous alkali metal or alkaline earth metal base with an isoxazoline derivative of the formula (IV) 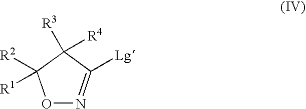
in which R¹, R², R³ and R⁴ are each as defined above for the formula (I) and Lg′ is a leaving group
to give the corresponding 3-arylmethylthio- or 3-heteroarylmethylthio-4,5-dihydroisoxazoline derivative of the formula (I).

2. The process as claimed in claim 1, wherein R¹ and R² are each independently (C₁-C₄)-alkyl, (C₂-C₃)-alkenyl, (C₂-C₃)-alkynyl, or (C₃-C₆)-cycloalkyl, and where each of the (C₁-C₄)-alkyl, (C₂-C₃)-alkenyl, (C₂-C₃)-alkynyl, or (C₃-C₆)-cycloalkyl radicals is optionally substituted by one or more radicals selected from the group consisting of halogen, cyano and (C₃-C₆)-cycloalkyl.

3. The process as claimed in claim 2, where R¹ and R² are each independently (C₁-C₄)-alkyl or (C₁-C₄)-haloalkyl.

4. The process as claimed in claim 2, wherein R¹ and R² are each independently methyl or ethyl each of which is optionally independently mono- or polyhalogenated.

5. The process as claimed in claim 4, wherein R¹ and R² are each independently methyl, ethyl or chloromethyl.

6. The process as claimed in claim 5, wherein R¹ and R² are each independently methyl or ethyl.

7. The process as claimed in claim 1, wherein R³ and R⁴ are each independently hydrogen or (C₁-C₄)-alkyl.

8. The process as claimed in claim 7, wherein R³ and R⁴ are both hydrogen.

9. The process as claimed in claim 1, wherein R⁵ is an unsubstituted or substituted aryl having from 6 to 10 carbon atoms or unsubstituted or substituted heteroaryl having from 3 to 5 carbon atoms and from 1 to 3 heteroatoms selected from the group consisting of N, O and S, where each of the aryl or heteroaryl radicals is optionally substituted by one or more identical or different radicals selected from the group consisting of halogen, cyano, ethyl, methyl, haloethyl, halomethyl, halomethoxy and haloethoxy.

10. The process as claimed in claim 9, wherein R⁵ is a substituted or unsubstituted phenyl, naphthyl, thienyl, furyl, pyrazolyl, pyridinyl, pyrimidinyl, pyrazinyl, pyridazinyl, imidazolyl, isothiazolyl, thiazolyl or oxazolyl optionally substituted by one or more identical or different radicals selected from the group consisting of halogen, cyano, ethyl, methyl, halomethoxy and halomethyl.

11. The process as claimed in claim 1, wherein the leaving group Lg is chlorine, bromine, iodine or a sulfonate group.

12. The process as claimed in claim 1, wherein the leaving group Lg′ is chlorine, bromine, or a sulfonate group, or a methylsulfonyl group.

13. The process as claimed in claim 1, wherein the leaving group Lg′ is chlorine.

14. The process as claimed in claim 1, wherein the compound of the formula (II) is reacted with an equimolar amount of the compound of the formula (IV) under phase transfer conditions with vigorous stirring.

15. The process as claimed in claim 14, wherein a biphasic system is used, comprising an aqueous, strongly basic alkali metal or alkaline earth metal hydroxide solution, and an organic phase which is an inert solvent or a mixture of inert solvents.

16. The process as claimed in claim 15, further comprising a phase transfer catalyst selected from the group consisting of a quaternary ammonium salt, a quaternary phosphonium salt, a crown ether, a cryptand and a polyethylene glycol.

17. The process as claimed in claim 14, wherein the reaction temperature is from −10° C. to 150° C.

18. The process as claimed in claim 17, wherein the compounds of formula (II) and formula (IV) are stirred vigorously under a protective gas atmosphere at a temperature of from 20° C. to 100° C.

19. The process as claimed in claim 11, wherein the sulfonate group is selected from the group consisting of methane-, trifluoromethane-, ethane-, benzene- and toluenesulfonate.

20. The process as claimed in claim 12, wherein the sulfonate group is selected from the group consisting of methane-, trifluoromethane-, ethane-, benzene- and toluenesulfonate.

21. The process as claimed in claim 14, wherein the compound of the formula (II) is obtained by crystallization and is used without further purification steps.

22. The process as claimed in claim 15, wherein the organic phase is an inert solvent selected from the group consisting of tetrahydrofuran, diethyl ether, acetonitrile, pentane, hexane, benzene, toluene, xylene, chlorobenzene, dichloromethane, chloroform, carbon tetrachloride, nitrobenzene and mixtures thereof.
